(19) United States
(12) Patent Application Publication
(10) Pub. No.: US 2010/0003289 A1
(43) Pub. Date: Jan. 7, 2010

(54) Controlled Release Complex Formulation For Oral Administration of Medicine For Diabetes and Method For The Preparation Thereof

(75) Inventors: Jong Soo Woo, Suwon-si (KR); Hong Gi Yi, Suwon-si (KR); Moon Hyuk Chi, Suwon-si (KR); Young Hun Kim, Suwon-si (KR)

Correspondence Address: SUGHRUE MION, PLLC, 2100 PENNSYLVANIA AVENUE, N.W., SUITE 800, WASHINGTON, DC, 20037, US

(73) Assignee: Hanmi Pharm, Co., Ltd., Hwaseong-gun (KR)

(21) Appl. No.: 11/722,560

(22) PCT Filed: Dec. 28, 2005
(86) PCT No.: PCT/KR2005/004609

§ 371 Date: Jun. 22, 2007

(30) Foreign Application Priority Data
Dec. 31, 2004 (KR) 10-2004-0117781

(51) Int. Cl. A61K 9/00; A61K 31/155; A61K 31/40; A61P 3/10

(52) U.S. Cl. 424/400; 514/635; 514/423

(57) ABSTRACT

A controlled release combination formulation for oral administration comprising a) a controlled release portion containing metformin or a pharmaceutically acceptable salt thereof as an active ingredient, and a combination of a polyethylene oxide and a natural gum as a carrier for controlled release; and b) a rapid-release portion containing a sulfonylurea-based medicine for treating diabetes as an active ingredient coated on the controlled release portion is useful for the treatment of diabetes, for it is capable of maintaining an effective concentration of the medicines in blood at a constant level.

### FIELD OF THE INVENTION

The present invention relates to an oral controlled release combination formulation of two medicines for diabetes and a method for the preparation thereof.

### BACKGROUND OF THE INVENTION

Metformin is an oral medication designed for helping control the patients' elevated blood sugar level by activating glucose receptor in the liver. It induces weight loss, reduces the level of blood-triglyceride and low-density lipoproteins (LDL), and increases high-density lipoproteins (HDL) in a diabetic patient. Therefore, it may be used as a primary drug for non-insulin-dependent diabetes mellitus (NIDDM).

Metformin in the tabletted form of its hydrochloride is currently marketed as GLUCOPHAGE® (Bristol-myers Squibb Company) and its daily dosage is determined individually on the basis of both effectiveness and tolerance, while not exceeding the maximum recommended dose of 2,550 mg per day. The side effects of metformin are loss of appetite, abdominal distension, nausea and diarrhea, while skin eruption or hives may break out on rare occasions. These side effects may be avoided by reducing the minimum and/or maintenance dose, or by administrating a controlled release formulation.

Glimepiride, one of the sulfonylureas for oral administration, has been used as a drug for non-insulin-dependent diabetic patients who can not improved by dietetic therapy, weight training and weight loss, and its tabletted form is marketed as AMARYL® (Aventis Pharmaceuticals Inc.).

Sulfonylurea-based medicines including glimepiride are known to react with β-cells to enhance insulin secretion and to exert long-term effects in reducing the blood-glucose level.

U.S. Pat. No. 6,031,004 discloses medication comprising a sulfonylurea derivative such as glyburide, glipizide and glimepride tabletted with a novel metformin salt for treating non-insulin-dependent diabetes; WO 00/03742 discloses a method for the manufacture of a combination formulation comprising (a) forming granules by wet granulation of a mixture of metformin and glibenclamide, (b) blending the granules with a tabletting aid and a diluent, (c) tabletting the blend, and (d) coating the obtained tablet with a hydrophilic cellulose polymer. However, this combination formulation shows the problem of unsatisfactory release behavior.

U.S. Pat. No. 6,682,759 discloses a method for the manufacture of a combination formulation comprising (a) tabletting metformin hydrochloride for controlled release using hydroxypropyl methylcellulose and polyethyleneoxide and (b) spraying on the resulting tablet glimepride dispersed in aqueous hydroxypropyl methylcellulose in the absence of a stabilizer. However, this combination formulation has the problem of reduced effective concentrations of the drugs because of the formation of drug derivatives; cyanoguanidine derivative of metformin and sulfonamide derivative of glimepride.

Therefore, there has been a continual need to develop improved controlled release formulation for oral administration of a combination of medicines for diabetes, which is capable of maintaining the effectiveness of the drug by uniform release over a prescribed period.

### SUMMARY OF THE INVENTION

Accordingly, it is an object of the present invention to provide a controlled release combination formulation for oral administration of metformin and a sulfonylurea-based antidiabetic medicine, which can be easily prepared and is capable of maintaining uniform release of the drugs over a long period of time, and a method for the preparation thereof.

In accordance with one aspect of the present invention, there is provided a controlled release combination formulation for oral administration comprising a) a controlled release portion containing metformin or a pharmaceutically acceptable salt thereof as an active ingredient, and a carrier for controlled release consisting of a polyethylene oxide and a natural gum; and b) a rapid-release portion coated on the controlled release portion containing a sulfonylurea-based antidiabetic medicine as an active ingredient.

### BRIEF DESCRIPTION OF THE DRAWINGS

The above and other objects and features of the present invention will become apparent from the following description of the invention taken in conjunction with the following accompanying drawings, which respectively show:

FIG. 1: a schematic diagram of the ingredients of the inventive controlled release combination formulation;

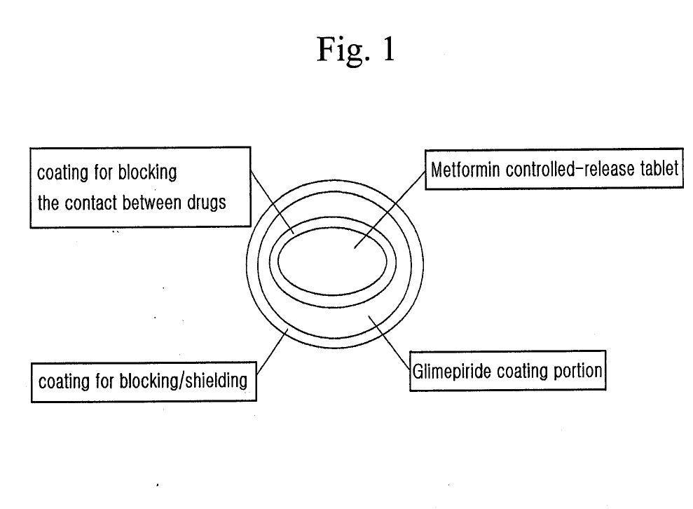

FIG. 2: in vitro drug release profiles of the controlled release tablets prepared in Examples 1 to 4 of the present invention, and a metformin-containing comparative formulation (GLUCOPHAGE® XR controlled release tablet, Bristol-Myers Squibb Company), respectively;

FIG. 3: in vitro drug release profiles of the controlled release tablets prepared in Examples 5 to 8 of the present invention and a comparative formulation (GLUCOPHAGE® XR controlled release tablet), respectively;

FIG. 4: in vitro drug release profiles of the controlled release tablets prepared in Examples 9 to 12 of the present invention and a comparative formulation (GLUCOPHAGE® XR controlled release tablet), respectively;

FIG. 5: in vitro drug release profiles of the controlled release tablets prepared in Example 12 of the present invention, the controlled release combination formulation prepared in Example 13 and a comparative formulation (GLUCOPHAGE® XR controlled release tablet, Bristol-Myers Squibb Company), respectively;

FIG. 6: in vitro drug release profiles of the controlled release combination formulation prepared in Example 13 of the present invention and a glimepiride-containing comparative formulation (AMARYL® tablet, Aventis Pharmaceuticals Inc), respectively;

FIG. 7: in vitro drug release profile of the controlled release tablet prepared in Example 12 of the present invention as function of the rotation rate of the release port;

FIG. 8: in vitro release profile of a comparative formulation (GLUCOPHAGE® XR controlled release tablet) as function of the rotation rate of the release port; and

FIG. 9: illustrating the stability of glimepiride as function of the solution pH.

### DETAILED DESCRIPTION OF THE INVENTION

The inventive controlled release combination formulation for oral administration comprise a) a controlled release portion containing metformin or a pharmaceutically acceptable salt thereof as an active ingredient, and a carrier for controlled release consisting of a polyethylene oxide and a natural gum; and b) a rapid-release portion coated on the controlled release portion containing a sulfonylurea-based antidiabetic medicine as an active ingredient.

Each ingredient of the inventive formulation is described in detail as follows:

### 1. Controlled Release Portion

The controlled release portion of the formulation of the present invention comprises an active ingredient, a carrier for controlled release, a pharmaceutically acceptable additive and a release-controlling agent. The amount of the controlled release portion may be in the range of 85 to 99.5% by weight based on the total weight of the formulation.

### (1) Active Ingredient for Controlled Release

The active ingredient of the controlled release portion is metformin, which is used for non-insulin-dependent diabetes mellitus, or its pharmaceutically acceptable salt, e.g., a chloride, succinate or fumarate.

### (2) Carrier for Controlled Release

The carrier for controlled release of the present invention is a combined mixture of a polyethylene oxide and a natural gum. The polyethylene oxide may have an average molecular weight of 100,000 to 7,000,000, or a mixture of two or more polyethylene oxides with different molecular weights may be also used.

Examples of the natural gum are xanthan gum, locust gum, guar gum, and a mixture thereof.

In accordance with the present invention, the weight ratio of the active ingredient and the carrier for controlled release may range from 1:0.01 to 1:1, and preferably, from 1:0.1 to 1:0.95. The polyethylene oxide:natural gum weight ratio may range form 1:0.1 to 1:10, preferably, from 1:0.5 to 1:5.

### (3) Pharmaceutically Acceptable Additive

The controlled release portion may further comprise pharmaceutically acceptable additives, and exemplary additives include a carrier acceptable for an oral solid formulation such as neutralized diluent carriers, binders, lubricants or a mixture thereof.

The neutralized diluent carrier may be lactose, dextrin, starch, microcrystallized cellulose, potassium phosphate monobasic, calcium carbonate, saccharide or silicon dioxide, and the like.

The binders of the present invention can be polyvinyl pyrrolidone or gelatin.

The lubricants of the present invention can be a zinc or magnesium salt of stearic acid and the like.

In addition, any conventional additive used in the pharmaceutical field for the preparation of an oral formulation may also be used.

In accordance with the present invention, the weight ratio of the active ingredient for controlled release:each of the pharmaceutically acceptable additives may range from 1:0.0005 to 1:0.3, preferably, from 1:0.001 to 1:0.1.

### (4) Release-Controlling Agent

In order to fine-control the release pattern of the active ingredient, a selective release-controlling agent such as a wax and a polyvinyl acetate/polyvinyl pyrrolidone mixture, which helps the carrier for controlled release in manifesting its gelling property in vivo, may be additionally used as an optional ingredient in the formulation of the present invention.

The active ingredient:the selective release-controlling agent weight ratio preferably ranges from 1:0 to 1:0.9, while the amount of said agent is preferably in the range of 0.001 to 0.1% by weight base on the total weight of the formulation.

### 2. Inner Coating Portion (Inner Separating Layer)

In order to prevent possible mutual interactions between the active ingredients of the controlled release portion and rapid-release portion so that the rapid release rate of the active ingredient of the rapid-release portion can be maintained undisrupted, the inventive controlled release combination formulation may further comprise an inner coating portion as an inner separating layer coated on the surface of the controlled release portion. The inner coating portion may be used in an amount ranging from 0.5 to 5% by weight based on the total weight of the formulation.

Representative examples of film-forming materials (a film-forming agent and a coating agent) used in the inner coating portion of the present invention include hydroxypropylmethylcellulose, hydroxypropylcellulose, hydroxyethylcellulose, cellulose acetatephthalate, ethylcellulose, methylcellulose, polymethacrylate, polyethylene glycol, talc, titanium dioxide, and a mixture thereof. In addition, any conventional additives used in the pharmaceutical field for the preparation of an oral solid formulation may also be used.

### 3. Rapid-Release Portion

In the formulation of the present invention, a rapid-release portion is coated on the surface of the controlled release portion, or on the surface of the inner coating portion if it is present. The rapid-release portion may comprise an active ingredient for rapid release, a stabilizer and a film-forming material and may be used in an amount ranging from 0.5 to 15% by weight based on the total weight of the formulation.

### (1) Active Ingredient for Rapid Release

The active ingredient of the rapid release portion is a sulfonylurea-based antidiabetic medicine such as glimepiride, glyburide, glipizide and gliclazide.

### (2) Stabilizer

In order to enhance the stability of the active ingredient, the rapid release portion may further comprise a stabilizer. Representative examples of the stabilizer include an antioxidant such as butylhydroxyanisole, butylhydroxytoluene and tocopherol; an inorganic base such as sodium hydroxide and ammonia; an organic base such as meglumine(N-methylglucamine), ethanolamine and propanolamine; a basic amino acid such as arginine, lysine and histidine, and the like. In addition, any conventional additives used in the pharmaceutical field for the preparation of an oral solid formulation may also be used. In accordance with the present invention, the active ingredient for rapid-release:stabilizer weight ratio may range from 1:0.01 to 1:1, preferably, from 1:0.1 to 1:0.5.

### (3) Film-Forming Material

The film-forming material used in the inner coating portion may also be used as the film-forming material of the rapid-release portion. The active ingredient for rapid-release:film-forming material weight ratio may range from 1:5 to 1:50, preferably, from 1:10 to 1:30.

### 4. Outer Coating Portion

In order to protect the combination formulation of the present invention from external influences, the inventive formulation may further comprise a film coating layer as an outer coating portion.

Film-forming materials (film-forming agents or coating agents) used in the outer coating portion may be the same as those used in the inner coating portion. The amount of the outer coating portion may be in the range of 0.5 to 5% by weight based on the total weight of the composition.

The controlled release combination formulation for oral administration may be prepared by a process comprising the steps of:

1) mixing metformin or a pharmaceutically acceptable salt thereof with a first hydrophilic carrier for controlled release and granulating the resulting mixture;

2) mixing the granules obtained in step 1 with a second hydrophilic carrier for controlled release, which is identical to or different from the first hydrophilic carrier;

3) adding a pharmaceutically acceptable additive to the mixture obtained in step 2 to prepare a controlled release portion;

4) coating the controlled release portion obtained in step 3 to prevent the possible interactions between the active ingredients of the final controlled release formulation; and

5) coating the coated controlled release formulation obtained in step 4 with a sulfonylurea-based antidiabetic medicine.

The method may further comprise the step of coating an outer coating portion.

The following Examples are intended to further illustrate the present invention without limiting its scope.

### EXAMPLES

### I. Preparation of Metformin Controlled Release Tablet

### Example 1

500 g of metformin.HCl (Hwail Pharm. Co., Ltd), 80 g of polyethylene oxide (Polyox® WSR Agglutinant, Molecular weight 5,000,000, Union Carbide) and 100 g of xanthan gum (Cpkelco) were each filtered through No. 30 mesh and mixed together. The mixture was placed in a high-speed mixer (SPG-2, Fujipaudal), and a binder solution made up of 20 g of polyvinyl pyrrolidone (Kollidon® K-90, BASF) dissolved in distilled water was added to the mixer, followed by mixing at a speed of 100˜1,000 rpm for 3 min to obtain granules. The granules were dried and filtered through No. 30 mesh. Thereafter, 200 g of a polyvinyl acetate/polyvinyl pyrrolidone mixture (Kollidon SR, BASF), 80 g of wax (Compritol® 888ATO, Gattefosse) and 10 g of silicon dioxide were added to the granules and mixed for 30 min. Finally, 10 g of magnesium stearate powder was added to the mixture, mixed for 3 min, and compressed to obtain a tablet having the composition of Table 1.

TABLE 1

| | | Content |
|---|---|---|
| Ingredients | | (wt %) |
| Granule | Metformin•HCl | 50 |
| forming | Polyethylene oxide | 8 |
| portion | (Polyox ® WSR, M.W 5,000,000) | |
| | Xanthan gum | 10 |
| | Polyvinyl pyrrolidone | 2 |
| Mixture | Polyvinyl acetate/Polyvinyl pyrrolidone | 20 |
| portion | mixture | |
| | Wax | 8 |
| | Silicon dioxide | 1 |
| | Magnesium stearate | 1 |

| | |
|---|---|
| Total | 100 |

### Examples 2 to 5

Tablets having the compositions listed in Tables 2 to 5 were prepared by repeating the procedure of Example 1 except for using Xanthan gum (Cpkelco) in the mixture portion or using polyethylene oxides having different molecular weights. In addition, the polyvinyl pyrrolidone binder was also excluded from the granule forming portion in these examples.

TABLE 2
Composition of a tablet of Example 2

| | | Content |
|---|---|---|
| Ingredients | | (wt %) |
| Granule | Metformin•HCl | 50 |
| forming | Polyethylene oxide | 5 |
| portion | (Polyox ® WSR, M.W 5,000,000) | |
| Mixture | Polyvinyl acetate/Polyvinyl pyrrolidone | 20 |
| portion | mixture | |
| | Wax | 13 |
| | Xanthan gum | 10 |
| | Silicon dioxide | 1 |
| | Magnesium stearate | 1 |

| | |
|---|---|
| Total | 100 |

TABLE 3
Composition of a tablet of Example 3

| | | Content |
|---|---|---|
| Ingredients | | (wt %) |
| Granule | Metformin•HCl | 50 |
| forming | Polyethylene oxide | 5 |
| portion | (Polyox ® N10, M.W 100,000) | |
| Mixture | Polyvinyl acetate/Polyvinyl pyrrolidone | 20 |
| portion | mixture | |
| | Wax | 13 |
| | Xanthan gum | 10 |
| | Silicon dioxide | 1 |
| | Magnesium stearate | 1 |

| | |
|---|---|
| Total | 100 |

TABLE 4
Composition of a tablet of Example 4

| | | Content |
|---|---|---|
| Ingredients | | (wt %) |
| Granule | Metformin•HCl | 50 |
| forming | Polyethylene oxide | 5 |
| portion | (Polyox ® 1105, M.W 900,000) | |
| Mixture | Polyvinyl acetate/Polyvinyl | 20 |
| portion | pyrrolidone mixture | |
| | Wax | 13 |
| | Xanthan gum | 10 |
| | Silicon dioxide | 1 |
| | Magnesium stearate | 1 |

| | |
|---|---|
| Total | 100 |

TABLE 5
Composition of a tablet of Example 5

| | | Content |
|---|---|---|
| Ingredients | | (wt %) |
| Granule | Metformin•HCl | 50 |
| forming | Polyethylene oxide | 10 |
| portion | (Polyox ® WSR, M.W 5,000,000) | |
| Mixture | Polyvinyl acetate/Polyvinyl | 20 |
| portion | pyrrolidone mixture | |
| | Wax | 8 |
| | Xanthan gum | 10 |
| | Silicon dioxide | 1 |
| | Magnesium stearate | 1 |

| | |
|---|---|
| Total | 100 |

### Example 6

A tablet having the composition shown in Table 6 was prepared by repeating the procedure of Example 1 except for not using the binder, polyvinyl pyrrolidone (Kollidon® K-90, BASF) binder during the granule formation step.

TABLE 6

| | | Content |
|---|---|---|
| Ingredients | | (wt %) |
| Granule | Metformin•HCl | 50 |
| forming | Polyethylene oxide | 10 |
| portion | (Polyox ® WSR, M.W 5,000,000) | |
| | Xanthan gum | 10 |
| Mixture | Polyvinyl acetate/Polyvinyl | 20 |
| portion | pyrrolidone mixture | |
| | Wax | 8 |
| | Silicon dioxide | 1 |
| | Magnesium stearate | 1 |

| | |
|---|---|
| Total | 100 |

### Example 7

A tablet having the composition shown in Table 7 was prepared by repeating the procedure of Example 1 except for using isopropyl alcohol in place of distilled water during the granule formation step.

TABLE 7

| | | Content |
|---|---|---|
| Ingredients | | (wt %) |
| Granule | Metformin•HCl | 50 |
| forming | Polyethylene oxide | 8 |
| portion | (Polyox ® WSR, M.W 5,000,000) | |
| | Xanthan gum | 10 |
| | Polyvinyl pyrrolidone | 2 |
| Mixture | Polyvinyl acetate/Polyvinyl | 20 |
| portion | pyrrolidone mixture | |
| | Wax | 8 |
| | Silicon dioxide | 1 |
| | Magnesium stearate | 1 |

| | |
|---|---|
| Total | 100 |

### Examples 8 to 10

Tablets having the compositions shown in Tables 8 to 10 were prepared by repeating the procedure of Example 1 except for using a distilled water/isopropyl alcohol mixture (1:1 (v/v)) in place of distilled water during the granule formation step.

TABLE 8
Composition of a tablet of Example 8

| | | Content |
|---|---|---|
| Ingredients | | (wt %) |
| Granule | Metformin•HCl | 50 |
| forming | Polyethylene oxide | 8 |
| portion | (Polyox ® WSR, M.W 5,000,000) | |
| | Xanthan gum | 10 |
| | Polyvinyl pyrrolidone | 2 |
| Mixture | Polyvinyl acetate/Polyvinyl | 28 |
| portion | pyrrolidone mixture | |
| | Silicon dioxide | 1 |
| | Magnesium stearate | 1 |

| | |
|---|---|
| Total | 100 |

TABLE 9
Composition of a tablet of Example 9

| | | Content |
|---|---|---|
| Ingredients | | (wt %) |
| Granule | Metformin•HCl | 50 |
| forming | Polyethylene oxide | 16 |
| portion | (Polyox ® WSR, M.W 5,000,000) | |
| | Xanthan gum | 10 |
| | Polyvinyl pyrrolidone | 2 |
| Mixture | Polyvinyl acetate/Polyvinyl | 20 |
| portion | pyrrolidone mixture | |
| | Silicon dioxide | 1 |
| | Magnesium stearate | 1 |

| | |
|---|---|
| Total | 100 |

TABLE 10
Composition of a tablet of Example 10

| | | Content |
|---|---|---|
| Ingredients | | (wt %) |
| Granule | Metformin•HCl | 50 |
| forming | Polyethylene oxide | 8 |
| portion | (Polyox ® WSR, M.W 5,000,000) | |
| | Xanthan gum | 18 |
| | Polyvinyl pyrrolidone | 2 |
| Mixture | Polyvinyl acetate/Polyvinyl | 20 |
| portion | pyrrolidone mixture | |
| | Silicon dioxide | 1 |
| | Magnesium stearate | 1 |

| | |
|---|---|
| Total | 100 |

### Example 11

A tablet having the composition shown in Table 11 was prepared by repeating the procedure of Example 1 except for using a distilled water/isopropyl alcohol mixture (1:1 (v/v)) during the granule formation step, as well as using xanthan gum (Cpkelco) and locust bean gum (Sigma) in the mixture portion.

TABLE 11

| | | Content |
|---|---|---|
| Ingredients | | (wt %) |
| Granule | Metformin•HCl | 50 |
| forming | Polyethylene oxide | 10 |
| portion | (Polyox ® WSR, M.W 5,000,000) | |
| | Polyvinyl pyrrolidone | 2 |
| Mixture | Polyvinyl acetate/Polyvinyl | 20 |
| portion | pyrrolidone mixture | |
| | Xanthan gum | 10 |
| | Locust bean gum | 6 |
| | Silicon dioxide | 1 |
| | Magnesium stearate | 1 |

| | |
|---|---|
| Total | 100 |

### Example 12

A tablet having the composition shown in Table 12 was prepared by repeating the procedure of Example 1 except for using a distilled water/isopropyl alcohol mixture (1:1 (v/v)) during the granule formation step, as well as using xanthan gum (Cpkelco) and locust bean gum (Sigma), without using the polyvinyl acetate/polyvinyl pyrrolidone mixture (Kollidon SR, BASF) in the mixture portion.

TABLE 12

| | | Content |
|---|---|---|
| Ingredients | | (wt %) |
| Granule | Metformin•HCl | 50 |
| forming | Polyethylene oxide | 10 |
| portion | (Polyox ® WSR, M.W 5,000,000) | |
| | Polyvinyl pyrrolidone | 2 |
| Mixture | Xanthan gum | 21 |
| portion | Locust bean gum | 15 |
| | Silicon dioxide | 1 |
| | Magnesium stearate | 1 |

| | |
|---|---|
| Total | 100 |

### II. Preparation of Metformin/Glimepiride Combination Formulation

### Example 13

The controlled release tablet of metformin obtained in Example 12 was coated in accordance with the following steps.

\textcircled{1} 20 g of hydroxypropyl methylcellulose (HPMC2910, Shin-Etsu) was dissolved in an ethanol/methylene mixture chloride (7/3 volume ratio), 2.7 g of polyethylene glycol 6000 (Sanyo chemical In.) was added thereto, and stirred to obtain a homogenous solution. The homogenous solution was filtered through No. 200 mesh and sprayed on the metformin controlled release tablet obtained in Example 12 to form a controlled release portion containing the metformin controlled release tablet.

\textcircled{2} 2.0 g of glimepiride (Cipla) was dissolved in an ethanol/methylene chloride mixture (7/3 volume ratio), 30 g of hydroxypropyl methylcellulose (HPMC2910, Shin-Etsu) was added thereto, and stirred until solubilized. 0.5 g of meglumine (N-methylglucamine, Sigma) and 4.0 g of polyethylene glycol 6000(Sanyo chemical In.) were added thereto and the resulting homogeneous solution was filtered through No. 200 mesh. Thereafter, the filtrate was sprayed on the release control portion containing metformin to form a film containing glimepiride thereon.

\textcircled{3} 20 g of hydroxypropyl methylcellulose (HPMC2910, Shin-Etsu) was dissolved in an ethanol/methylene chloride mixture (7/3 volume ratio) and 2.4 g of titanium dioxide (Kronos International) was added thereto. The mixture was then granulated in a homogenizing grinder, 2.7 g of polyethylene glycol 6000 (Sanyo chemical In.) was added to the resulting mixture to obtain a homogenous solution, which was filtered through No. 200 mesh. The filtrate was then sprayed onto the glimepiride film-coated controlled release tablet of metformin to obtain a combination formulation having the composition shown in Table 13.

TABLE 13

| | | | Content |
|---|---|---|---|
| Ingredients | | | (wt %) |
| Controlled | Granule | Metformin•HCl | 46.11 |
| release | forming | Polyethylene oxide | 9.22 |
| portion | portion | (Polyox ® WSR, M.W 5,000,000) | |
| | | Polyvinyl pyrrolidone | 1.84 |
| | Mixture | Xanthan gum | 19.37 |
| | portion | Locust bean gum | 13.83 |
| | | Silicon dioxide | 0.92 |
| | | Magnesium stearate | 0.92 |

| | | |
|---|---|---|
| Inner coating | Hydroxypropylmethyl cellulose | 1.85 |
| portion | Polyethyleneglycol 6000 | 0.25 |
| Rapid-release | Glimepiride | 0.18 |
| portion | Hydroxypropylmethyl cellulose | 2.77 |
| | Polyethyleneglycol 6000 | 0.37 |
| | Meglumine | 0.05 |
| Coat portion | Hydroxypropylmethyl cellulose | 1.85 |
| | Polyethyleneglycol 6000 | 0.25 |
| | Titanium dioxide | 0.22 |

| | |
|---|---|
| Total of tablets | 100 |

### Example 14

A combination formulation having the composition shown in Table 14 was prepared by repeating the procedure of Example 13 except for using 0.5 g of butylhydroxyanisole in place of meglumin as a stabilizer for the rapid-release portion.

TABLE 14

| | | | Content |
|---|---|---|---|
| Ingredients | | | (wt %) |
| Controlled | Granule | Metformin•HCl | 46.11 |
| release | forming | Polyethylene oxide | 9.22 |
| portion | portion | (Polyox ® WSR, M.W 5,000,000) | |
| | | Polyvinyl pyrrolidone | 1.84 |
| | Mixture | Xanthan gum | 19.37 |
| | portion | Locust bean gum | 13.83 |
| | | Silicon dioxide | 0.92 |
| | | Magnesium stearate | 0.92 |

| | | |
|---|---|---|
| Inner coating | Hydroxypropylmethyl cellulose | 1.85 |
| portion | Polyethyleneglycol 6000 | 0.25 |
| Rapid-release | Glimepiride | 0.18 |
| portion | Hydroxypropylmethyl cellulose | 2.77 |
| | Polyethyleneglycol 6000 | 0.37 |
| | Butylhydroxyanisole | 0.05 |
| Coat portion | Hydroxypropylmethyl cellulose | 1.85 |
| | Polyethyleneglycol 6000 | 0.25 |
| | Titanium dioxide | 0.22 |

| | |
|---|---|
| Total of tablets | 100 |

### Example 15

A combination formulation having the composition shown in Table 15 was prepared by repeating the procedure of Example 13 except for using 0.5 g of tocopherol (Roche, Switzerland) in place of meglumin as the stabilizer for the rapid release portion.

TABLE 15

| | | | Content |
|---|---|---|---|
| Ingredients | | | (wt %) |
| Controlled | Granule | Metformin•HCl | 46.11 |
| release | forming | Polyethylene oxide | 9.22 |
| portion | portion | (Polyox ® WSR, M.W 5,000,000) | |
| | | Polyvinyl pyrrolidone | 1.84 |
| | Mixture | Xanthan gum | 19.37 |
| | portion | Locust bean gum | 13.83 |
| | | Silicon dioxide | 0.92 |
| | | Magnesium stearate | 0.92 |

| | | |
|---|---|---|
| Inner coating | Hydroxypropylmethyl cellulose | 1.85 |
| portion | Polyethyleneglycol 6000 | 0.25 |
| Rapid-release | Glimepiride | 0.18 |
| portion | Hydroxypropylmethyl cellulose | 2.77 |
| | Polyethyleneglycol 6000 | 0.37 |
| | Tocopherol | 0.05 |
| Coat portion | Hydroxypropylmethyl cellulose | 1.85 |
| | Polyethyleneglycol 6000 | 0.25 |
| | Titanium dioxide | 0.22 |

| | |
|---|---|
| Total of tablets | 100 |

### Comparative Example 1

A combination formulation having the composition shown in Table 16 was prepared from the controlled release tablet of metformin prepared in Example 12 by repeating the film coating procedure of Example 13 except for not using the meglumine stabilizer.

TABLE 16

| | | | Content |
|---|---|---|---|
| Ingredients | | | (wt %) |
| Controlled | Granule | Metformin•HCl | 46.13 |
| release | forming | Polyethylene oxide | 9.23 |
| portion | portion | (Polyox ® WSR, M.W 5,000,000) | 1.84 |
| | | Polyvinyl pyrrolidone | |
| | Mixture | Xanthan gum | 19.38 |
| | portion | Locust bean gum | 13.84 |
| | | Silicon dioxide | 0.92 |
| | | Magnesium stearate | 0.92 |

| | | |
|---|---|---|
| Inner coating | Hydroxypropylmethyl cellulose | 1.85 |
| portion | Polyethyleneglycol 6000 | 0.25 |
| Rapid-release | Glimepiride | 0.18 |
| portion | Hydroxypropylmethyl cellulose | 2.77 |
| | Polyethyleneglycol 6000 | 0.37 |
| Coat portion | Hydroxypropylmethyl cellulose | 1.85 |
| | Polyethyleneglycol 6000 | 0.25 |
| | Titanium dioxide | 0.22 |

| | |
|---|---|
| Total of tablets | 100 |

### Test Example 1

### In Vitro Release Test 1

In order to examine the effects of natural gum and polyethylene oxide as carriers for controlled release on the release rate of the drug, the tablets prepared in Examples 1 to 12 were subjected together with GLUCOPHAGE® XR controlled release tablet (Bristol-Myers Squibb Company) as a comparative formulation to in vitro release tests in accordance with the release test method described in Korea pharmacopoeia (the paddle method). The release pattern of metformin.HCl from each of the tablets was measured under the following conditions.
Release test apparatus: Erweka DT 80 (Erweka, Germany)
Release solution: The 2nd solution for the disintegrating-test described in Korea pharmacopoeia (artificial gastric fluid)
Release solution temperature: 37±0.5° C.
Amount of the release solution: 900 mL
Rotation rate: 50 rpm
Sampling times: Aliquots of the release solution were collected at 1, 2, 3, 4, 6, 8, and 10 hrs, filtered through a 0.45 μm membrane, and used as test samples. After each sampling of the release solution, the release-test system was refilled with an equal amount of fresh release solution.
Analyzing method: The absorbances of a sample and a standard solution were measured at 233 nm employing distilled water as a reference to calculate the corresponding release ratio.
Calculation of the released amount: Cumulative release amount

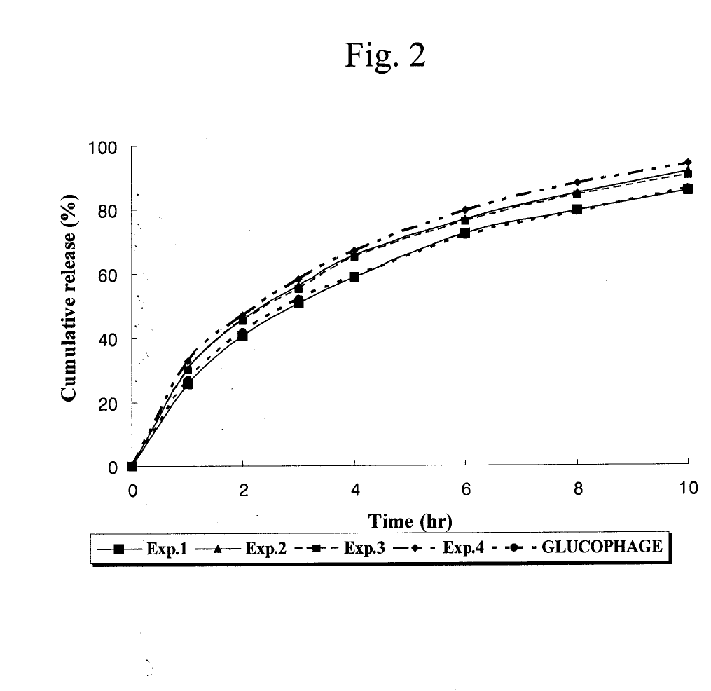

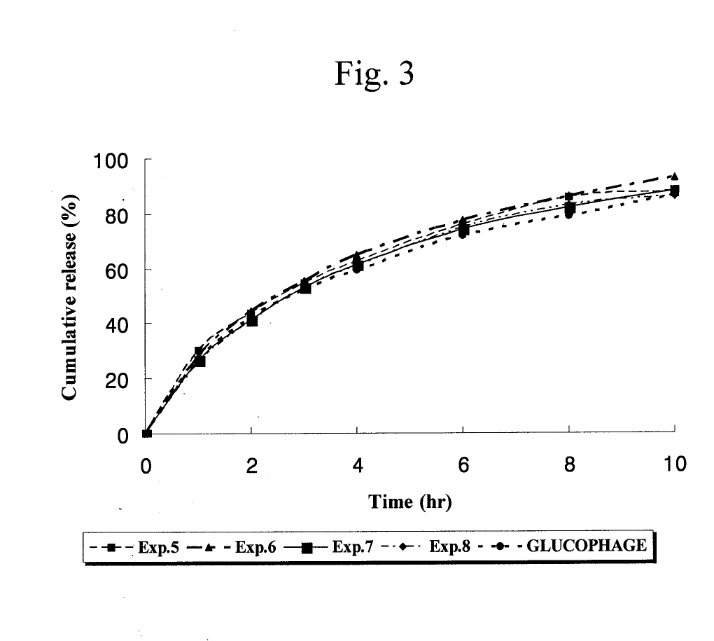

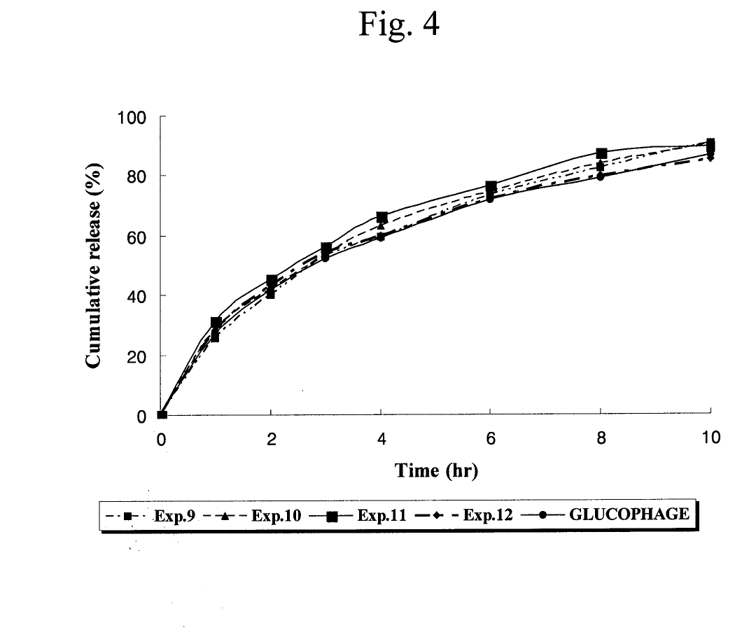

As can be seen from FIGS. 2 to 4, the release rate becomes slow as the amount of polyethylene oxide or the natural gum increases. Especially, the tablet of Example 12 releases the drug continuously in a release pattern similar to that of the comparative formulation.

### Test Example 2

### In Vitro Release Test 2

In order to examine how the film coating of the controlled release tablet obtained in Example 13 affect the release rates of the drugs, in vitro release-tests were conducted by repeating the method of Test Example 1 except for using the controlled release formulation prepared in Example 12, the combination formulation prepared in Example 13, and GLUCOPHAGE® XR controlled release tablet as a comparative formulation.

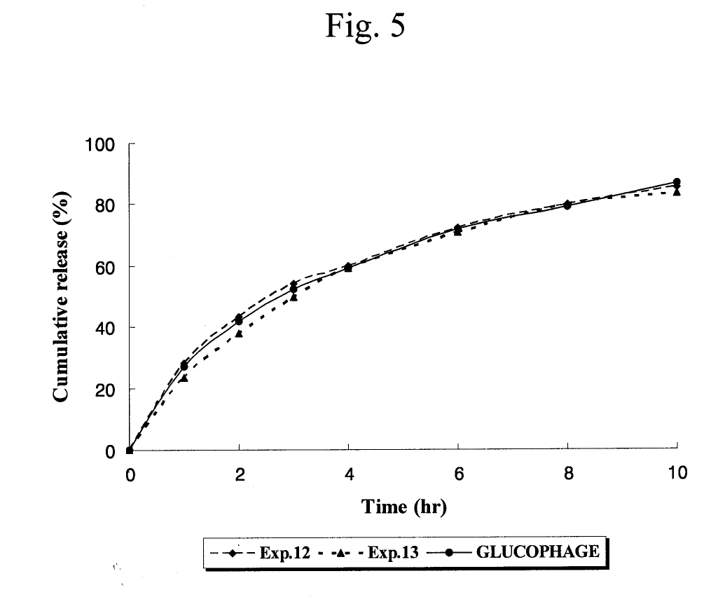

As can be seen from FIG. 5, the controlled release combination formulation of Example 13 shows a continuous drug release pattern similar to those of the combination formulation of Example 12 and the comparative formulation.

### Test Example 3

### In Vitro Release Test 3

In order to examine how the glimepiride coating of the controlled release combination formulation affect the release rate, the controlled release combination formulation prepared in Example 13 and Amaryl tablet (Aventis Pharmaceuticals Inc.) as a comparative formulation were subjected to in vitro release tests in accordance with the release test method described in Korea pharmacopoeia (the paddle method). The release pattern of the active glimepiride ingredient from each of the formulations was measured under the following conditions.
Release test apparatus: Erweka DT 80 (Erweka, Germany)
Release solution: Phosphate buffer solution (pH 7.8)
Release solution temperature: 37±0.5° C.
Amount of the release solution: 900 mL
Rotation rate: 75 rpm
Sampling times: Aliquots of the release solution were collected at 5, 10, 15 and 30 mins, filtered through a 0.45 μm membrane, and used as test samples. After each sampling of the release solution, the release-test system was refilled with an equal amount of fresh release solution.
Analyzing method: The release ratios of a sample and a standard solution were calculated in accordance with the Liquid Chromatograph method described in The Korea pharmacopoeia under the following conditions.
   Column: Octadecyl silylated column
   Mobile phase: After mixing sodium dihydrogen phosphate, acetonitrile and water (0.5 g:500 ml:500 ml) together, pH of the mixture was adjusted to 2.5 to 3.5 with 20% by volume of phosphate.
   Detector: UV Spectrophotometer (measuring wavelength 228 nm)
   Amount of injection: 50 μl
   Flow rate: 0.5 ml/min
Calculation of released amount: Cumulative release amount

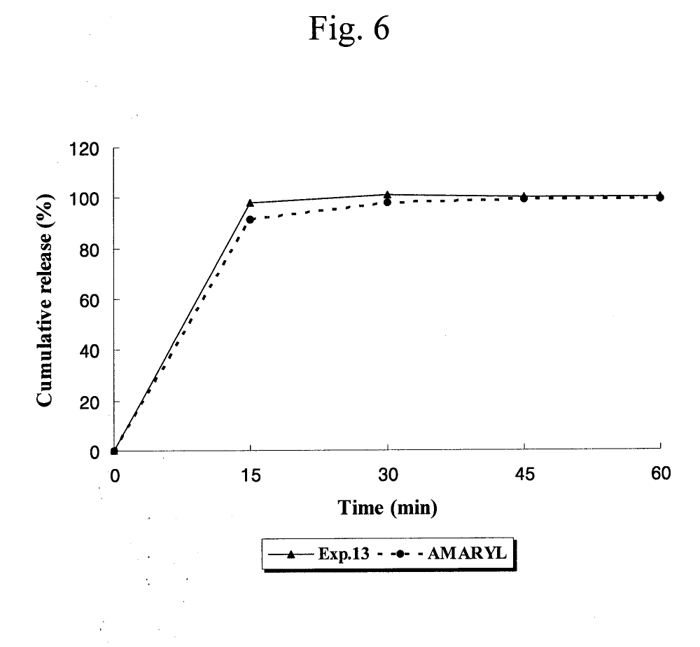

As can be seen from FIG. 6, the release rate of glimepiride from the formulation of Example 13 was equivalent to that of the comparative Amaryl tablet formulation.

### Test Example 4

### In Vitro Release Test 4

In vitro release tests were conducted for the tablet prepared in Example 12 and the comparative formulation by repeating the method of Test Example 1, except for adjusting the rotation rate to 100 rpm and 150 rpm.

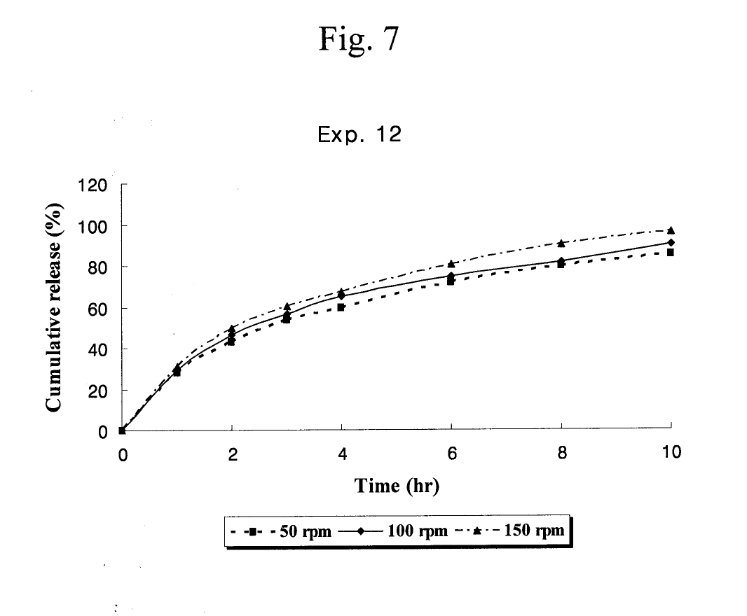

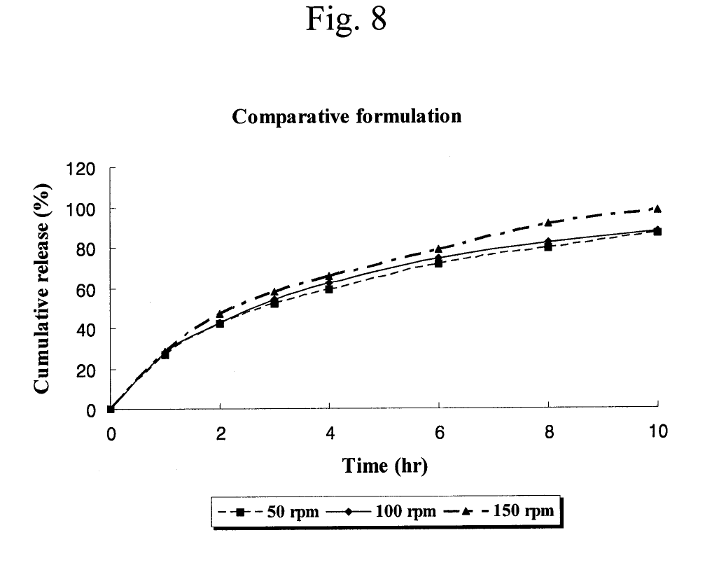

As can be seen from FIGS. 7 and 8, the tablet of Example 12 displays a steady release pattern, without initial burst release of the drug even at a high rotation rate.

### Test Example 5

### Stability Test

In order to examine the stability of glimepiride in a solution as function of pH changes, only the glimepiride rapid-release portion of the formulation of Example 13 was separated, and dissolved in each of the solutions listed in Table 17. Each of the resulting solutions was kept at room temperature and the glimepiride contents thereof were measured at predetermined times.

TABLE 17

| | | Rate | | |
|---|---|---|---|---|
| | | constant | | |
| Solution | | (K) | Log K | T_{50%} |
| pH 1.2 | The 1^{st} solution of the | 1.534 | 0.186 | 0.45 |
| | disintegrating-test in Korea | | | |
| | pharmacopoeia | | | |
| pH 4.0 | British pharmacopoeia buffer | 0.337 | −0.472 | 2.06 |
| | solution (1998) | | | |
| pH 6.8 | The 2^{nd} solution of disintegrating- | 0.126 | −0.900 | 5.50 |
| | test in Korea pharmacopoeia | | | |
| pH 7.8 | Amaryl release solution as a | 0.065 | −1.187 | 10.66 |
| | comparative formulation | | | |
| | (phosphate buffer solution (pH 7.8) | | | |
| pH | Meglumin 1% solution | 0.002 | −2.721 | 346.50 |
| 10.0 | | | | |

T_{50%}: the time taken for 50% disintegration of the drug (T_{50%} = 0.693/K)

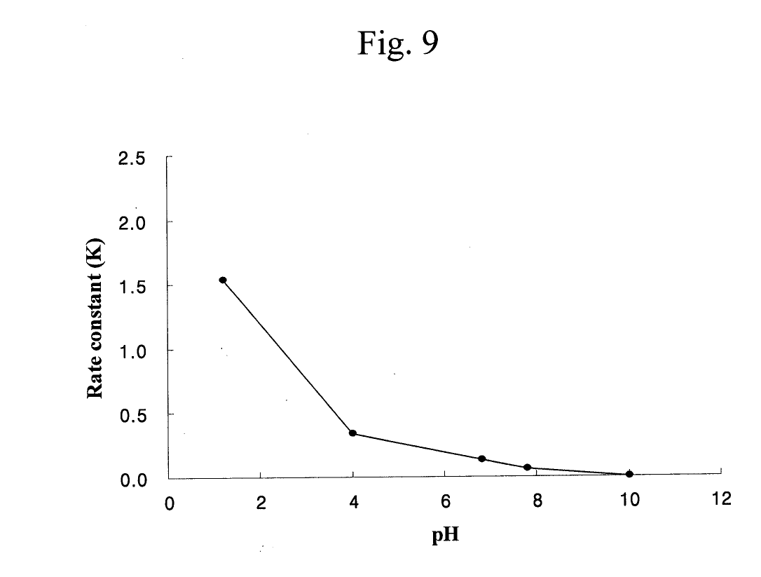

As can be seen from FIG. 9, it was found that in the solution containing meglumin which is an alkaline compound, glimepiride was most stable as judged by its lowest K value.

### Test Example 6

### Stability Test (Accelerated Test (40° C., Relative Humidity 75%)

In order to examine the effect of meglumin, an organic base, on the stability of glimepiride, a stability test was conducted by employing the controlled release combination formulations of Example 13 and Comparative Example 1, and the results are shown in Table 18.

TABLE 18

Glimepiride decomposition product
(sulfonamide (%), standard 2.5% or less

| | | After 1 | After 3 | After 6 |
|---|---|---|---|---|
| | Beginning | month | months | months |
| Example 13 | Not-detected | 0.07 | 0.29 | 1.04 |
| Comparative | 0.10 | 0.30 | 0.73 | 4.00 |
| Example 1 | | | | |

As shown in Table 18, a large amount of sulfonamides, the main decomposition product of glimepiride, was detected for the formulation of Comparative Example 1 which does not contain meglumin, after 6 months under the accelerated condition. Therefore, it was confirmed that, when meglumine was not added to the formulation, the glimepiride stability becomes poor and, its effective concentration becomes lower.

While the invention has been described with respect to the above specific embodiments, it should be recognized that various modifications and changes may be made and also fall within the scope of the invention as defined by the claims that follow.

1. A controlled release combination formulation for oral administration comprising a) a controlled release portion containing metformin or a pharmaceutically acceptable salt thereof as an active ingredient, and a carrier for controlled release consisting of a polyethylene oxide and a natural gum; b) an inner coating portion coated on the surface of the controlled release portion, and c) a rapid-release portion coated on the inner coating portion containing a sulfonylurea-based antidiabetic medicine as an active ingredient and a stabilizer.

2. The controlled release combination formulation of claim 1, wherein the amount of the controlled release portion is 85 to 99.5% by weight and the amount of the rapid-release portion is 0.5 to 15% by weight based on the total weight of the formulation.

3. The controlled release combination formulation of claim 1, wherein the amount of the inner coating portion is 0.5 to 5% by weight based on the total weight of the formulation.

4. The controlled release combination formulation of claim 1, which further comprises an outer coating portion to protect the controlled release combination formulation from external influences.

5. The controlled release combination formulation of claim 4, wherein the amount of the outer coating portion is 0.5 to 5% by weight based on the total weight of the formulation.

6. The controlled release combination formulation of claim 1, wherein the pharmaceutically acceptable salt of metformin is metformin chloride, metformin succinate or metformin fumarate.

7. The controlled release combination formulation of claim 1, wherein the polyethylene oxide has an average molecular weight in the range of 100,000 to 7,000,000.

8. The controlled release combination formulation of claim 1, wherein the natural gum is selected from the group consisting of xanthan gum, locust bean gum, guar gum and a mixture thereof.

9. The controlled release combination formulation of claim 1, wherein the metformin:carrier for controlled release weight ratio ranges from 1:0:01 to 1:1.

10. The controlled release combination formulation of claim 1, wherein the controlled release portion further comprises a pharmaceutically acceptable additive and a release-controlling agent.

11. The controlled release combination formulation of claim 10, wherein the pharmaceutically acceptable additive is a neutralized diluent carrier, binder, lubricant or a mixture thereof.

12. The controlled release combination formulation of claim 10, wherein the release-controlling agent is a wax or a polyvinyl acetate/polyvinyl pyrrolidone mixture.

13. The controlled release combination formulation of claim 1, wherein the inner coating portion-forming material is selected from the group consisting of hydroxylmethylcellulose, hydroxypropyicellulose, hydroxyethylcellulose, cellulose acetatephthalate, ethylcellulose, methylcellulose, polymethacrylate, polyethylene glycol, talc, titanium dioxide and a mixture thereof.

14. The controlled release combination formulation of claim 1, wherein the sulfonylurea-based antidiabetic medicine is selected from the group consisting of glimepiride, glyburide plipizide and gliclazide.

15. The controlled release combination formulation of claim 1, wherein the stabilizer is selected from the group consisting of an antioxidant, an inorganic base, an organic base and a basic amino acid.

16. The controlled release combination formulation of claim 1, wherein the sulfonylurea-based antidiabetic medicine:stabilizer weight ratio ranges from 1:0.01 to 1:1.

17. The controlled release combination formulation of claim 4, wherein the material forming the outer coating portion is selected from the group consisting of hydroxypropylmethylcellulose, hydroxypropylcellulose, hydroxyethylcellulose, cellulose acetatephthalate, ethylcellulose, methylcellulose, polymethacrylate, polyethylene glycol, talc, titanium dioxide and a mixture thereof.

18. A method for preparing the controlled release combination formulation of claim 1 comprising:
1) mixing metformin or a pharmaceutically acceptable salt thereof with a first hydrophilic carrier for controlled release and granulating the resulting mixture;
2) mixing the granules obtained in step 1 with a second hydrophilic carrier for controlled release, which is identical to or different from the first hydrophilic carrier;
3) adding a pharmaceutically acceptable additive to the mixture obtained in step 2 to prepare a controlled release portion;
4) coating the surface of controlled release portion obtained in step 3 with an inner coating portion-forming material to obtain a coated controlled release formulation; and
5) coating the coated controlled release formulation obtained in step 4 with a sulfonylurea-based antidiabetic medicine and a stabilizer.

19. The method of claim 18, which further comprises a step of coating an outer coating portion to protect the controlled release combination formulation from external influences.

20-21. (canceled)
